(19) United States
(12) Patent Application Publication
(10) Pub. No.: US 2025/0066276 A1
(43) Pub. Date: Feb. 27, 2025

(54) CATALYSTS AND METHODS FOR CONVERSION OF 1,1,2-TRICHLORO-1,2,2-TRIFLUOROETHANE (CFC-113) TO 1,1,2-TRIFLUOROETHANE (HFC-143)

(71) Applicant: Honeywell International Inc., Charlotte, NC (US)

(72) Inventors: Akbar Mahdavi-Shakib, Buffalo, NY (US); Haiyou Wang, Amherst, NY (US)

(21) Appl. No.: 18/807,095

(22) Filed: Aug. 16, 2024

Related U.S. Application Data
(60) Provisional application No. 63/534,000, filed on Aug. 22, 2023.

(51) Int. Cl. C07C 17/25; C07C 17/354

(52) U.S. Cl. CPC C07C 17/25; C07C 17/354; C07C 2523/44; C07C 2523/56

(57) ABSTRACT

HFO-1132 and, in particular, HFO-1132E, may be produced from 1,1,2-trichloro-1,2,2-trifluoroethane (CFC-113). In a first step, 1,1,2-trifluoroethane (HFC-143) is produced by hydrogenating 1,1,2-trichloro-1,2,2-trifluoroethane (CFC-113) by reaction with hydrogen in the presence of a catalyst to produce 1, 1,2-trifluoroethane (HFC-143). The highly exothermic hydrogenation step may be moderated by diluting the catalyst and/or by diluting the 1, 1,2-trichloro-1,2,2-trifluoroethane (CFC-113) feedstock. The 1, 1,2-trifluoroethane (HFC-143) may then be dehydrofluorinated in the presence of a catalyst to produce trans-1,2-difluoroethylene (HFO-1132E) and/or cis-1,2-difluoroethylene (HFO-1132Z). The cis-1,2-difluoroethylene (HFO-1132Z) may then be isomerized to produce trans-1,2-difluoroethylene (HFO-1132E).

### CROSS-REFERENCE TO RELATED APPLICATIONS

The present application claims the benefit under title 35, U.S.C. section 119(e) of U.S. Provisional Patent Application Ser. No. 63/534,000, entitled “Catalysts and methods for conversion of 1,1,2-trichloro-1,2,2-trifluoroethane (CFC-113) to 1,1,2-trifluoroethane (HFC-143)”, filed on Aug. 22, 2023, the entire disclosure of which is expressly incorporated by reference herein.

### FIELD

The present disclosure is directed to catalysts and methods for producing trans-1,2-difluoroethylene (HFO-1132E) from 1,1,2-trichloro-1,2,2-trifluoroethane (CFC-113) and, in particular, to catalysts and methods for converting 1,1,2-trichloro-1,2,2-trifluoroethane (CFC-113) to a 1,1,2-trifluoroethane (HFC-143) intermediate in a process to produce trans-1,2-difluoroethylene (HFO-1132E).

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of 1,2-difluoroethylene (HFO-1132) include refrigerants, either used alone or in blends with other components, solvents for organic materials, and as a chemical intermediate in the synthesis of other halogenated hydrocarbon solvents.

Improved methods for the production of 1,2-difluoroethylene (HFO-1132) and, in particular, trans-1,2-difluoroethylene (HFO-1132E), are desired.

### SUMMARY

The production of trans-1,2-difluoroethylene (HFO-1132E) from 1,1,2-trichloro-1,2,2-trifluoroethane (CFC-113) involves a multi-step catalytic process.

In a first step, 1,1,2-trifluoroethane (HFC-143) is produced by hydrogenating 1,1,2-trichloro-1,2,2-trifluoroethane (CFC-113) by reaction with hydrogen in the presence of a catalyst to produce 1,1,2-trifluoroethane (HFC-143). The 1,1,2-trifluoroethane (HFC-143) may then be dehydrofluorinated in the presence of a catalyst to produce trans-1,2-difluoroethylene (HFO-1132E) and/or cis-1,2-difluoroethylene (HFO-1132Z). The cis-1,2-difluoroethylene (HFO-1132Z) may then be isomerized to produce trans-1,2-difluoroethylene (HFO-1132E).

The present disclosure provides catalysts and methods for controlling the highly exothermic reaction of the first step above, namely, producing 1,1,2-trifluoroethane (HFC-143) by hydrogenating 1,1,2-trichloro-1,2,2-trifluoroethane (CFC-113) by reaction with hydrogen in the presence of a catalyst.

In a first approach, the catalyst used in the foregoing reaction may be diluted with a diluent material which functions as a thermally absorbing medium to aid in managing heat generated during the reaction.

In a second approach, the 1,1,2-trichloro-1,2,2-trifluoroethane (CFC-113) reactant, prior to or during the reaction, may be combined with an amount of a feedstock diluent, such as a non-reactive gas, and/or a feedstock diluent in the form of an organic molecule such as 1,1,2-trifluoroethane (HFC-143), for example, which may be a separate product from the reaction itself or introduced independently into the reaction from an external source.

Each of the above approaches, when employed separately or in combination with each other, may increase the selectivity towards the desired product, 1,1,2-trifluoroethane (HFC-143) and/or selectivity to desired intermediates, and/or decrease the selectivity to undesired by products.

In one form thereof, the present disclosure provides a method for producing 1,1,2-trifluoroethane (HFC-143), comprising: hydrogenating 1,1,2-trichloro-1,2,2-trifluoroethane (CFC-113) by reaction with hydrogen in the presence of a catalyst to produce 1,1,2-trifluoroethane (HFC-143), the catalyst comprising: a catalytic material comprising from 0.1 to 1.0 wt. % of a catalytic metal supported on a support, based on a total weight of the catalytic metal and the support; and a diluent material, wherein the amount of catalytic material is from 5 to 70 volume percent, based on the total volume of the catalytic material and diluent material.

In the above method, the support may be alpha alumina.

In another form thereof, the present disclosure provides a palladium metal catalyst useful for hydrogenating 1,1,2-trichloro-1,2,2-trifluoroethane (CFC-113) by reaction with hydrogen to produce 1,1,2-trifluoroethane (HFC-143), the catalyst comprising: a catalytic material comprising from 0.1 to 1.0 wt. % of palladium metal supported on an alumina (Al₂O₃) support, based on a total weight of the catalytic metal and the support; and a diluent material, wherein the amount of catalytic material is from 5 to 70 volume percent, based on the total volume of the catalytic material and diluent material.

In the above catalyst, the support may be alpha alumina.

In a further form thereof, the present disclosure provides a method for producing 1,1,2-trifluoroethane (HFC-143), including combining 1,1,2-trichloro-1,2,2-trifluoroethane (CFC-113) with a feedstock diluent to form a reaction mixture; and reacting the 1,1,2-trichloro-1,2,2-trifluoroethane (CFC-113) in the reaction mixture with hydrogen in the presence of a catalyst to produce a product mixture.

### BRIEF DESCRIPTION OF THE DRAWINGS

FIG. 1 is a schematic diagram of the apparatus used in Example 1 for the conversion of 1,1,2-trichloro-1,2,2-trifluoroethane (CFC-113) to 1,1,2-trifluoroethane (HFC-143).

FIG. 2 is a graph showing the overall selectivity of the 0.2% Pd/alpha Al₂O₃ catalyst toward ‘HFC-143+ recyclables’ as a function of catalyst concentration and temperature for the experiment in Example 2.

FIG. 3 is a graph showing the % selectivity to undesired by-products as a function of phase of the alumina support in 200-210° C. range for the experiment in Example 4.

FIG. 4 is a graph showing substrate conversion for the Pd/Al₂O₃ catalysts with alpha, theta, and delta alumina supports for the experiment in Example 4.

FIG. 5 is a graph showing percent conversion of the 1,1,2-trichloro-1,2,2-trifluoroethane (CFC-113) substrate for the 0.2% Pd/alpha Al₂O₃ catalysts as a function of contact time at three different temperatures for the experiment in Example 5.

FIG. 6 is a graph showing product selectivity for the conversion of 1,1,2-trichloro-1,2,2-trifluoroethane (CFC-113) to 1,1,2-trifluoroethane (HFC-143) using 0.2% Pd/alpha Al₂O₃ catalysts as a function of temperature for the experiment in Example 5.

FIG. 7 is a graph showing product selectivity HFC-143, HCFC-123a, and HCFC-133b in the process of 1,1,2-trichloro-1,2,2-trifluoroethane (CFC-113) conversion to 1,1,2-trifluoroethane (HFC-143) using 0.2% Pd/alpha Al₂O₃ catalysts as a function of temperature for the experiment in Example 5.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the names for refrigerants including the ASHRAE number, such as “R-143”, the IUPAC name “1,1,2-trifluoroethane”, and the type-number abbreviation, such as “HFC-143”, may all be used interchangeably to refer to the same refrigerants.

As used herein, catalytic material refers to the metal catalyst as well as any catalyst support material that is used with the metal catalyst.

As used herein, catalyst dilution refers to a method of reducing a concentration of a catalyst in a chemical reaction by combining a catalytic material with a diluent substance which is inert to the reaction or does not catalyze the reaction.

### II. Overview

The present disclosure generally relates to a method for producing E-1,2-difluoroethylene (HFO-1132E) from 1,1,2-trichloro-1,2,2-trifluoroethane (CFC-113) according to a three-step process shown below (“Process 1”), which includes the following three steps: (i) hydrogenating 1,1,2-trichloro-1,2,2-trifluoroethane (CFC-113) to produce 1,1,2-trifluoroethane (HFC-143), (ii) dehydrofluorinating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E).

Schematic equations for the three steps of Process 1 are represented below:

Process 1

CFCl₂—CF₂Cl (CFC-113)+3H₂→CFH₂—CF₂H (HFC-143)+3 HCl

ΔH_{f}=−59.7 kcal/mol (i)

CFH₂—CF₂H→x trans-CFH═CHF (HFO-1132E)+(1−x) cis-CFH═CFH (HFO-1132Z)+HF (ii)

cis-CFH═CFH (HFO-1132Z)→trans-CFH═CHF (HFO-1132E) (iii)

The heat of reaction for Step (i) was calculated using DFT (Density Functional Theory) method. The ΔH_{f} of −59.7 kcal/mol indicates that step (i) is a strongly exothermic reaction step.

It has been found that the first step involving the conversion of 1,1,2-trichloro-1,2,2-trifluoroethane (CFC-113) to 1,1,2-trifluoroethane (HFC-143) may be improved by employing methods to control the large exotherm generated in the exothermic reaction. In a first approach, the hydrogenation catalyst may be diluted to manage the heat produced by the reaction and the reaction rate. In a second approach, the 1,1,2-trichloro-1,2,2-trifluoroethane (CFC-113) reactant, prior to or during the reaction, may be diluted with an amount of a feedstock diluent. The foregoing dilution methods allow for better control of the exotherm in step (i) and minimization of catalyst deactivation and formation of undesired by-products. Details of the catalyst dilution, feedstock dilution, and associated conditions are provided in Section III below.

Further details regarding each of steps (i), (ii), (iii) are also set forth below.

### III. Step (i)

Step (i) of the process for producing 1,2-difluoroethylene (HFO-1132) involves hydrogenating 1,1,2-trichloro-1,2,2-trifluoroethane (CFC-113) to produce 1,1,2-trifluoroethane (HFC-143). While catalysis may enhance the reaction rate and overall efficiency, the exothermic nature of the hydrogenation reaction itself may lead to several disadvantages. It has been found that the high temperatures in the hydrogenation reaction in step (i) may lead to catalyst deactivation and rapid loss of catalyst activity. In addition, the increased heat and energy in the reaction environment may promote undesired side reactions. Each of the foregoing may reduce the selectivity towards the desired product, 1,1,2-trifluoroethane (HFC-143).

To overcome these challenges, the present disclosure provides catalyst and feedstock dilution methods for improving the product selectivity for the 1,1,2-trichloro-1,2,2-trifluoroethane (CFC-113) to 1,1,2-trifluoroethane (HFC-143) conversion.

### A. Catalyst Dilution

The present methods may be used to control the rate of the reaction and prevent excessive catalytic deactivation. By diluting the catalyst, the reaction rate may be carefully tuned to prevent the formation of unwanted side products and enhance the lifespan of the catalyst.

### (i) Catalysts

The catalyst and diluent material play an important role in the reaction. Specifically, in the hydrogenation step, the catalytic material may comprise a catalytic metal such as palladium, platinum, rhodium, ruthenium, iron, cobalt or nickel.

The catalytic metal may be supported on a support such as activated carbon, porous aluminosilicate (ca. zeolites), alumina, silica, titania, zirconia, zinc oxide, aluminum fluoride, and the like. The alumina may be alpha alumina, delta alumina, theta alumina, or gamma alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried, calcined, and reduced to form active metal catalyst.

The catalytic material may comprise the catalytic metal supported on a support in an amount as low as about 0.01 wt. %, about 0.05 wt. %, about 0.1 wt. %, about 0.2 wt. %, about 0.3 wt. %, about 0.4 wt. %, or as high as about 0.5 wt. %, about 0.6 wt. %, about 0.7 wt. %, about 0.8 wt. about 0.9 wt. %, about 1 wt. %, about 1.5 wt. %, about 2 wt. %, about 5 wt. %, about 10 wt. %, about 20 wt. %, about 30 wt. %, about 40 wt. %, about 50 wt. %, or within any range encompassed by any two of the foregoing values as endpoints, based on the total weight of the catalytic metal and the support. For supported noble metal catalysts such as Pd and Pt, the metal loading may be ranged from about 0.01 wt. % to about 5 wt. %, preferably from about 0.05 wt. % to about 2 wt. %, and more preferably from about 0.1 wt. %, to about 1 wt. %.

When a palladium catalyst is used, the loading of palladium on the support, such as an alpha alumina support, may be from about 0.01 wt. % to about 5 wt. %, preferably from about 0.05 wt. % to about 2 wt. %, and more preferably from about 0.1 wt. % to about 1 wt. %.

The catalyst used in step (i) may have a proper BET (Brunauer, Emmet, and Teller) surface area. In some embodiments, the BET surface area of the catalyst may be as low as about 1 m²/g, about 3 m2/g, about 5 m²/g, about 10 m²/g, about 15 m2/g, about 20 m2/g 2, about 30 m2/g, about 40 m²/g, about 50 m2/g, about 100 m²/g, about 200 m²/g, or as high as about 250 m2/g, about 300 m²/g, about 400 m²/g, about 500 m²/g, about 600 m²/g, about 700 m²/g m2, about 800 m²/g, about 900 m²/g, about 1000 m²/g, about 2000 m²/g, or within any range encompassed by any of the foregoing values as endpoints. For alumina supported metal catalysts, the BET surface area may be from about 1 m²/g to about 500 m²/g, preferably from about 1 m²/g to about 200 m2/g, more preferably from about 1 m²/g to about 100 m²/g, and most preferably from about 1 m²/g to about 20 m2/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

When a palladium catalyst is used on an alpha alumina support, the BET surface area may be from about 1 m²/g to about 500 m²/g, preferably from about 1 m²/g to about 200 m²/g, more preferably from about 1 m²/g to about 100 m²/g, and most preferably from about 1 m²/g to about 20 m2/g.

### (ii) Catalyst Pretreatment

The catalyst may be pretreated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints. As part of the catalyst pretreatment, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

When a palladium catalyst is used on an alpha alumina support, the catalyst may be dried at a temperature of from about 200° C. to about 700° C., preferably from about 200° C. to about 500° C., most preferably from about 200° C. to about 300° C.

When a palladium catalyst is used on an alpha alumina support, the catalyst may be exposed to an inert gas such as N₂ for from about 1 hour to about 20 hours, preferably from about 1 hour to about 10 hours, most preferably, from about 1 hour to about 3 hours.

(iii) Catalyst Compositions

The present disclosure also includes catalyst compositions, such as those utilized in the diluted catalyst hydrogenation process described herein.

In one embodiment, the catalyst composition comprises a palladium metal catalyst useful for hydrogenating 1,1,2-trichloro-1,2,2-trifluoroethane (CFC-113) by reaction with hydrogen to produce 1,1,2-trifluoroethane (HFC-143), the catalyst including a catalytic material comprising from 0.1 to 1.0 wt. % of palladium metal supported on an alumina (Al₂O₃) support, preferably an alpha alumina support, based on a total weight of the catalytic metal and the support; and a diluent material, wherein the amount of catalytic material is from about 5 to about 70 volume percent, based on the total volume of the catalytic material and diluent material.

The diluent material may comprise an inert substance which is not itself reactive in the step (i) reaction, such as a metal, a metal alloy, a metal mesh, glass beads, inert alpha alumina, or carbon black. The metal mesh material may be made of stainless steel or nickel alloy such as Monel, Inconel, and the like. These meshes have a relatively large surface area and provide physical support for the catalyst, allowing the reaction mixture to flow through while reducing the catalyst's concentration.

The diluent material may be combined with the catalytic material by solids mixing techniques, such as simple solid mixing or shaking to evenly combine and distribute the catalytic material and the diluent material.

The diluent material may be present in an amount such that the amount of catalytic material may be as low as about 5 volume percent, about 10 volume percent, about 15 volume percent, about 20 volume percent, about 25 volume percent, or as high as about 30 volume percent, about 35 volume percent, about 40 volume percent, about 45 volume percent, about 50 volume percent, about 55 volume percent, about 60 volume percent, about 65 volume percent, about 70 volume percent, or within any range encompassed by any two of the foregoing values as endpoints, based on the total volume of the catalytic material and diluent material. For example, the amount of catalytic material may be from about 5 volume percent to about 70 volume percent, from about 10 volume percent to about 50 volume percent, or from about 10 volume percent to about 30 volume percent, based on the total volume of the catalytic material and diluent material.

The hydrogenation reaction of Step (i) may be carried out in the gas or vapor phase in a suitable reactor, for example a tubular reactor made from a material which is resistant to temperature and/or corrosion such as nickel and its alloys, including Hastelloy (for example, Hastelloy C276), Inconel (for example, Inconel 600). Incoloy, and Monel, and the vessels may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Products may be flowed through one or more scrubbers to remove by-products from the reaction, such as hydrogen fluoride (HF) and/or hydrogen chloride (HCl), and the reaction products may be collected by capture in a cooled cylinder, for example.

### B. Feedstock Dilution

To mitigate the heat of the reaction, prevent excessive catalytic deactivation, and/or minimize the formation of undesired by-products, the 1,1,2-trichloro-1,2,2-trifluoroethane (CFC-113) feedstock may be diluted with a feedstock diluent such as an inert gas or one or more non-reactive organic molecules, each of which do not participate in the underlying reaction. With feedstock dilution, either undiluted catalyst may be used, or diluted catalyst may be used as described above.

Suitable inert gases include nitrogen, argon, and the like.

Non-reactive organic molecules that may be used as feedstock diluents may include internal feedstock diluents, which are organic molecules that are produced in the step (i) reaction and then optionally separated from other products and recycled or otherwise reintroduced into the step (i) reaction by combining with the 1,1,2-trichloro-1,2,2-trifluoroethane (CFC-113) feedstock.

For example, suitable non-reactive organic molecules include 1,1,2-trifluoroethane (HFC-143) which is the target product of the step (i) reaction and may be separated from the step (i) product mixture before being combined with the 1,1,2-trichloro-1,2,2-trifluoroethane (CFC-113) feedstock. Alternatively, 1,1,2-trifluoroethane (HFC-143) feedstock diluent may be introduced independently from a source other than the products of the step (i) reaction.

Other non-reactive organic molecules that may be used as feedstock diluents may include external feedstock diluents, which are organic molecules that are not produced in the step (i) reaction but rather are introduced from an external source into the step (i) reaction by combining with the 1,1,2-trichloro-1,2,2-trifluoroethane (CFC-113) feedstock.

Non-reactive organic molecules that may be used as external feedstock diluents may include fluoromethane (HFC-41), difluoromethane (HFC-32), trifluoromethane (HFC-23), 1,1-difluoroethane (HFC-152a), 1,1,1,2-tetrafluoroethane (HFC-134a), 1,1,1,2,2-pentafluoroethane (HFC-125), 1,1,1,2-tetrafluoropropane (HFC-254eb), 1,1,1,2-tetrafluoropropane (HFC-254eb), 1,1,1,3,3-pentafluoropropane (HFC-245fa), 1,1,1,2,2-pentafluoropropane (HFC-245cb), 1,1,1,2,3-pentafluoropropane (HFC-245eb), 1,1,1,2,3,3-hexafluoropropane (HFC-236ea), 1,1,1,3,3,3-hexafluoropropane (HFC-236fa), 1,1,1,2,3,3,3-heptafluoropropane (HFC-227ea), and combinations of the foregoing.

The mole ratio of feedstock diluent to 1,1,2-trichloro-1,2,2-trifluoroethane (CFC-113) may range from 0.25/1 to 10/1, preferably from 0.5/1 to 8/1, and more preferably from 1/1 to 4/1.

### C. Step (i) Reaction

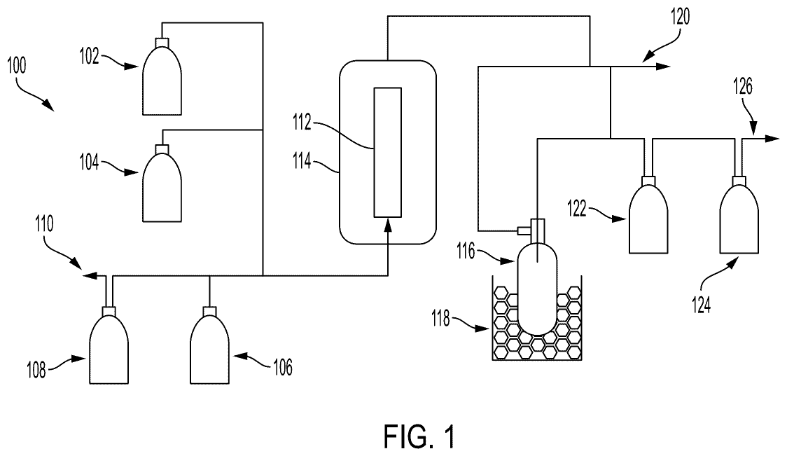

One embodiment of a reactor apparatus suitable for the reaction in step (i) is provided in FIG. 1. Referring to the process flow diagram 100 shown therein, a supply of N₂, which acts as an inert carrier, is provided from cylinder 102 and a supply of H₂ is provided from cylinder 104. The organic feedstock which may comprise 1,1,2-trichloro-1,2,2-trifluoroethane (CFC-113), is supplied from cylinder 106. Cylinders 102, 104 and 106 are all connected to KOH scrubber 108 which is coupled to vent 110. The supply of N₂, H₂, and organic feedstock is fed into reactor 112 which is surrounded by box oven 114. The reaction may be monitored by taking samples from outlet 120 and conducting a GC analysis.

As the reaction proceeds, the products are fed into holding tank 116 which is immersed in a bath of dry ice or dry ice and acetone mixture bath 118. The dry ice or dry ice and acetone mix bath is configured to hold the temperature of holding tank 116 at about −87° C. Holding tank 116 is coupled to buffer knock-out tank 112 to prevent potential backflow of KOH solution, which is also coupled to KOH scrubber 124. Scrubber 124 has a vent 126 which opens to the atmosphere. The materials which comprise target product 1,1,2-trifluoroethane (HFC-143), recyclable by-products such as 1-chloro-1,1,2-trifluoroethane (HCFC-133b), and 1,2-dichloro-1,1,2-trifluoroethane (HCFC-123a), acids such as HCl and HF, non-recyclable by-products such as 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), 1-chloro-1,2-difluoroethane (HCFC-142a), and chloroethane (HCC-160), and unconverted raw material 1,1,2-trichloro-1,2,2-trifluoroethane (CFC-113) collected in holding tank 116 may be subjected a number purification steps such as acid removal, drying, and distillation to isolate HFC-143 product. A portion of purified HFC-143 can be co-fed to Step (I) reactor as a diluent.

The reaction temperature may be as low as about 100° C., about 150° C., about 200° C., about 210° C., about 220° C., about 230° C., about 240° C., about 250° C., about 260° C., about 270° C., about 280° C., about 290° C., or as high as about 300° C., about 350° C., about 400° C., or within any range encompassed by two of the foregoing values as endpoints, such as from about 100° C. to about 300° C., or preferably from about 150° C. to about 250° C., for example.

When a palladium catalyst is used on an alpha alumina support, the reaction temperature may be from about 100° C. to about 400° C., preferably from about 100° C. to about 300° C., most preferably from about 150° C. to about 250° C.

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, or within any range encompassed by two of the foregoing values as endpoints. For example, the contact time may be preferably from about 1 second to about 60 seconds.

When a palladium catalyst is used on an alpha alumina support, the contact time may be from about 1 second to about 60 seconds, preferably from about 5 seconds to about 40 seconds, most preferably from about 10 seconds to about 30 seconds.

The pressure may be as little as about 1 psig, about 3 psig, about 5 psig, about 10 psig, about 15 psig, about 20 psig, about 30 psig, about 35 psig or about 40 psig, or as great as about 50 psig, about 60 psig, about 70 psig, about 80 psig, about 90 psig, about 100 psig, about 120 psig, about 150 psig, about 200 psig or about 250 psig, about 300 psig, or within any range encompassed by two of the foregoing values as endpoints. For example, the pressure may be preferably from about 10 to about 100 psig.

When a palladium catalyst is used on an alpha alumina support, the pressure may be from about 1 psig to about 300 psig, preferably from about 1 psig to about 200 psig, most preferably from about 10 psig to about 100 psig.

The mole ratio of hydrogen to CFC-113 reactants may be as little about 2:1, about 3:1, about 4:1, about 5:1, about 5.5:1 or as great as about 6:1, about 6.5:1, about 7.5:1 or about 8:1, about 12:1, about 15:1, or about 20:1, or within any range encompassed by two of the foregoing values as endpoints. The mole ratio of hydrogen to CFC-113 may be preferably from 3:1 to 15:1, and more preferably from 4:1 to 10:1.

When a palladium catalyst is used on an alpha alumina support, the mole ratio of hydrogen to CFC-113 reactants may be from about 2:1 to about 20:1, preferably from 3:1 to 15:1, most preferably from 4:1 to 10:1

As demonstrated by the Examples herein, the hydrogenation step may achieve a selectivity to the 1,1,2-trifluoroethane (HFC-143) product of as little as about 10%, about 20%, about 30%, about 40%, about 50%, about 60%, about 70% to as great as about 75%, about 80%, about 90%, or greater, for example, or within any range encompassed by two of the foregoing values as endpoints.

When a palladium catalyst is used on an alpha alumina support, the hydrogenation step may achieve a selectivity to the 1,1,2-trifluoroethane (HFC-143) product of about 10% to about 70%, preferably about 10% to about 50%, most preferably from about 20% to about 40%.

The hydrogenation reaction may also produce several intermediates such as 1-chloro-1,1,2-trifluoroethane (HCFC-133b), and 1,2-dichloro-1,1,2-trifluoroethane (HCFC-123a), and trifluoroethylene (HFO-1123). These intermediates are recyclable and can be eventually converted to 1,1,2-trifluoroethane (HFC-143). As demonstrated by the Examples herein, the hydrogenation step may achieve a combined selectivity to 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, greater than about 40%, greater than about 50%, greater than about 60%, greater than about 70%, greater than about 80%, greater than about 90%, greater than about 95%, or within any range encompassed by two of the foregoing values as endpoints.

When a palladium catalyst is used on an alpha alumina support, the hydrogenation step may achieve a combined selectivity and/or selectivity to each of 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, preferably greater than about 40%, most preferably greater than about 50%. The hydrogenation reaction may also produce several by-products such as 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), 1-chloro-1,2-difluoroethane (HCFC-142a), and chloroethane (HCC-160) which are the result of dehydrofluorination side reactions. These by-products are undesirable as they are difficult to recycle or convert to 1,1,2-trifluoroethane (HFC-143). As demonstrated by the Examples herein, the hydrogenation step may achieve a combined selectivity to 1,1-difluoroethane (HFC-152a), ethane (HC-170), 1,1,1-trifluoroethane (HFC-143a) 1-chloro-1,2-difluoroethane (HCFC-142a), and chloroethane (HCC-160) of less than about 30%, less than about 25%, less than about 20%, less than about 15%, less than about 10%, less than about 9%, less than about 8%, less than about 7%, less than about 6%, less than about 5%, or within any range encompassed by two of the foregoing values as endpoints.

When a palladium catalyst is used on an alpha alumina support, the hydrogenation step may achieve a combined selectivity and/or selectivity to each of to 1,1-difluoroethane (HFC-152a), ethane (HC-170), 1,1,1-trifluoroethane (HFC-143a) 1-chloro-1,2-difluoroethane (HCFC-142a), and chloroethane (HCC-160) of less than about 30%, preferably less than about 25%, most preferably less than about 20%.

As also demonstrated by the Examples herein, the hydrogenation step may achieve a conversion of 1,1,2-trichloro-1,2,2-trifluoroethane (CFC-113) to 1,1,2-trifluoroethane (HFC-143) from as little as about 10%, about 20%, about 30%, about 40%, about 50%, about 60%, about 70% or about 75% to as great as about 90%, about 95%, about 97%, about 98%, about 99% or greater, for example, or within any range encompassed by two of the foregoing values as endpoints.

When a palladium catalyst is used on an alpha alumina support, the hydrogenation step may achieve a conversion of 1,1,2-trichloro-1,2,2-trifluoroethane (CFC-113) to 1,1,2-trifluoroethane (HFC-143) of greater than about 10%, preferably greater than about 20%, most preferably greater than about 30%.

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed hydrogen treatment at temperatures of from about 100° C. to about 400° C., preferably from about 200° C. to about 350° C. for carbon and alumina supported transition metal catalysts.

When a palladium catalyst is used on an alpha alumina support, one or more of the following properties may be present. The reaction temperature may be from about 100° C. to about 400° C., preferably from about 100° C. to about 300° C., most preferably from about 150° C. to about 250° C. The contact time may be from about 1 second to about 60 seconds, preferably from about 5 seconds to about 40 seconds, most preferably from about 10 seconds to about 30 seconds. The pressure may be from about 1 psig to about 300 psig, preferably from about 1 psig to about 200 psig, most preferably from about 10 psig to about 100 psig. The mole ratio of hydrogen to CFC-113 reactants may be from about 2:1 to about 20:1, preferably from 3:1 to 15:1, most preferably from 4:1 to 10:1 The hydrogenation step may achieve a selectivity to the 1,1,2-trifluoroethane (HFC-143) product of about 10% to about 70%, preferably about 10% to about 50%, most preferably from about 20% to about 40%. The hydrogenation step may achieve a combined selectivity and/or selectivity to each of 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, preferably greater than about 40%, most preferably greater than about 50%. The hydrogenation step may achieve a combined selectivity and/or selectivity to each of to 1,1-difluoroethane (HFC-152a), ethane (HC-170), 1,1,1-trifluoroethane (HFC-143a) 1-chloro-1,2-difluoroethane (HCFC-142a), and chloroethane (HCC-160) of less than about 30%, preferably less than about 25%, most preferably less than about 20%. The hydrogenation step may achieve a conversion of 1,1,2-trichloro-1,2,2-trifluoroethane (CFC-113) to 1,1,2-trifluoroethane (HFC-143) of greater than about 10%, preferably greater than about 20%, most preferably greater than about 30%.

D. Combining Catalyst Dilution with Feedstock Dilution

In the hydrogenation reaction of step (i), the techniques of catalyst dilution and feedstock dilution, as described above and in the Examples below, may be combined, namely, may be used concurrently or in combination with each other.

### IV. Step (ii)

The dehydrofluorination reaction of Step (ii) may be carried out in the gas or vapor phase in a suitable reactor, for example a tubular reactor made from a material which is resistant to temperature and/or corrosion such as nickel and its alloys, including Hastelloy (for example, Hastelloy C276), Inconel (for example Inconel 600), Incoloy, and Monel wherein the vessels which may be lined with fluoropolymers.

The reactor may be first cleaned and flushed with an inert gas such as nitrogen, followed by packing with a catalyst such as those described below. The catalyst may be preteated within the reactor such as by drying in the manner described further below, followed by metering the reactants into the rector to initiate the reaction.

The process flow may be in the down or up direction through a bed of the catalyst. Reactants may be flowed through a scrubber to remove by-products from the reaction, such as hydrogen fluoride (HF) and/or hydrogen chloride (HCl), and the reaction products may be collected by capture in a cooled cylinder, for example.

The catalyst and process conditions play an important role in the dehydrofluorination reaction.

Suitable catalysts for the dehydrofluorination reaction include metal oxides such as chromium oxide, aluminum oxide, iron oxide, and magnesium oxide. Fluorination treatment of the catalyst may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrofluorination reaction include metal fluorides such as chromium fluoride, alumina fluoride, iron fluoride, magnesium fluoride, and various combinations of thereof.

Other metals, such as Pd and Ni, may also be loaded onto the above fluorinated metal oxides, for example, via a wet impregnation process wherein a salt of the metal is exposed to the fluorinated metal oxide support in solution, followed by drying, calcination, and then reduction with hydrogen gas.

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, based on a total weight of the catalyst and support. For supported noble metal (Pd, Pt, etc.) catalyst, the metal loading may be ranged from 0.01 to 5 wt. %, preferably from 0.05 to 2 wt. %, and more preferably from 0.1 to 1 wt. %.

The catalyst used in step (ii) may have a proper BET (Brunauer, Emmet, and Teller) surface area. In some embodiments, the BET surface area of the catalyst may be as low as 10 m²/g, 20 m2/g, 30 m2/g, 40 m2/g, 50 m2/g, 60 m2/g, 70 m2/g, 80 m2/g, 90 m2/g, 100 m2/g, or as high as 110 m2/g, 120 m2/g, 130 m2/g, 140 m2/g, 150 m2/g, 175 m2/g, 200 m2/g, 225 m2/g, 250 m2/g, 300 m2/g, or within any range encompassed by any of the foregoing values as endpoints. For metal oxides catalysts, the BET surface area may be preferably greater than 100 m²/g. For fluorinated metal oxides catalysts, the BET surface area may be preferably greater than 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

The catalyst may also be pretreated by drying at elevated temperatures, as low as about 200° C., about 250° C., about 300° C., about 350° C., about 360° C., about 370° C., or as high as about 380° C., about 390° C., about 400° C., about 450° C., about 500° C., or within any range encompassed by two of the foregoing values as endpoints. As part of the catalyst pretreatment, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

The temperature range for dehydrofluorination reaction may be as low as about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints. The temperature may be preferably from about 250° C. to about 450° C., and more preferably from about 300° C. to about 400° C.

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints. For example, the pressure may be from about 1 psig to about 50 psig, preferably from about 5 psig to about 30 psig, and more preferably from about 10 psig to about 20 psig.

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be from about 1 second to about 60 seconds.

In the dehydrofluorination reactions of Step (ii), the cis/trans molar ratio of the 1,2-difluoroethylene in the product mixture may be as low as about 1, about 2, about 3, about 4, about 5, about 6, or as high as about 7, about 8, about 9, about 10, about 11, about 12, about 13, about 14, about 15 or within any range encompassed by two of the foregoing values as endpoints. For example, the cis/trans ratio may be from about 2 to about 15.

The selectivity to the desired 1,2-difluoroethylene product (the sum of 1232E and 1232Z) may be as low as about 80%, about 85%, about 90%, about 91%, about 92%, about 93%, about 94%, about 95% or as high as about 96%, about 97%, about 98%, about 99%, or within any range encompassed by two of the foregoing values as endpoints. For example, the selectivity may be from about 89% to about 99%.

The conversion of the starting material to 1,2-difluoroethylene may be as low as about 10%, about 20%, about 30%, about 40%, or as high as about 50%, about 60%, about 70%, about 80%, about 90%, about 95%, about 99%, or within any range encompassed by two of the foregoing values as endpoints.

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C., for fluorinated catalysts or hydrogen treatment at temperatures of from about 100° C. to about 400° C., preferably from about 200° C. to about 350° C. for supported transition metal catalysts.

During the reaction, by-products formed in the step (i) and/or step (ii) reactions, such as HCFC-133b, HCFC-1133, HCFC-123a, HFO-1123 may be recycled back to the reactor input as desired.

Additionally, the present process advantageously avoids and/or minimizes formation of 1,1,1,-trifluoroethane (HFC-143a) wherein the products of Step (ii), including trans-1,2-difluoroethylene (HFO-1132E), may include less than 5 wt. %, less than 3 wt. %, less than 1 wt. %, less than 0.5 wt. %, or less than 0.1 wt. % of 1,1,1,-trifluoroethane (HFC-143a), based on a total weight of the product composition.

### V. Step (iii)

The 1,2-difluoroethylene (HFO-1132) obtained in step (ii) above may be produced as a mixture containing both the trans-1,2-difluoroethylene (HFO-1132E) and cis-1,2-difluoroethylene (HFO-1132Z) isomers.

In step (iii), the cis-1,2-difluoroethylene (HFO-1132Z) isomer may be converted to the trans-1,2-difluoroethylene (HFO-1132E) isomer either by exposure to heat and/or a catalyst to yield a final product comprising, consisting or, or consisting of, the trans-1,2-difluoroethylene (HFO-1132E) isomer in high purity, such as at least about 95 wt. %, at least about 99.0 wt. %, at least about 99.9 wt. %, at least about 99.99 wt. % or greater.

The isomerization reaction may be conducted in any suitable reaction vessel or reactor, but it should preferably be constructed from materials which are resistant to corrosion such as nickel and its alloys, including Hastelloy (for example, Hastelloy C276), Inconel (for example Inconel 600), Incoloy, and Monel wherein the vessels which may be lined with fluoropolymers. These may be single pipe or multiple tubes packed with an isomerization catalyst

The temperature range for the isomerization reaction may be as low as about 100° C. about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints.

The reaction may be conducted at atmospheric pressure, super-atmospheric pressure or under vacuum. The vacuum pressure can be from about 5 torr to about 760 torr. Contact time of the reactants with the catalyst may range from about 0.5 seconds to about 120 seconds, however, longer or shorter times can be used.

### VI. Reaction Products

The multi-step reaction described in Sections I.-V. above may yield a composition which comprises trans-1,2-difluoroethylene (HFO-1132E) in relatively high purity.

In one embodiment, the composition may comprise trans-1,2-difluoroethylene (HFO-1132E) present in an amount of at least 95 wt. %; and 1,1,1,-tritfluoroethane (HFC-143a) present in an amount of less than 5 wt. %, based on a total weight of the composition.

In another embodiment, the composition may comprise trans-1,2-difluoroethylene (HFO-1132E) present in an amount of at least 97 wt. %; and 1,1,1,-tritfluoroethane (HFC-143a) present in an amount of less than 3 wt. %, based on a total weight of the composition.

In another embodiment, the composition may comprise trans-1,2-difluoroethylene (HFO-1132E) present in an amount of at least 99 wt. %; and 1,1,1,-tritfluoroethane (HFC-143a) present in an amount of less than 1 wt. %, based on a total weight of the composition.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### EXAMPLES

### Example 1

### Catalyst Dilution Study

This Example demonstrates beneficial effect of catalyst dilution in improving product selectivity when using Pd/Al₂O₃ as the catalyst for the conversion of 1,1,2-trichloro-1,2,2-trifluoroethane (CFC-113) to 1,1,2-trifluoroethane (HFC-143). The experimental apparatus used for this example is shown in FIG. 1. The set up includes a feed system containing gas flow controllers for N₂ and H₂ and a Micromotion mass flow meter connected to a research control valve (RCV) controlling the organic flow rate. The reactor consists of a one-inch SS tube packed with the catalyst. A thermocouple is inserted into the middle of the catalyst bed to read the operating temperature. The pressure control system consists of a RCV which is controlling the pressure by getting feedback from the pressure transducer placed after the reactor. For GC analysis, samples are taken after the reactor using a sample bag filled with 50 ml of water to capture HCl and HF. Before the GC analysis, the sample bag is heated at 60° C. for one hour to assure that all the organic content is in the gas phase. Then, a sample is taken using a syringe and injected into the GC instrument for analysis.

In the first example, 0.5% Pd/theta Al₂O₃ catalyst was diluted with 40 ml of ⅛″ SS mesh packing material and then loaded into the reactor. The catalyst was pretreated with H₂ at 200° C. for one hour. Then, while 150 ml/min of H₂ was passing through the catalyst bed at 200° C., 1,1,2-trichloro-1,2,2-trifluoroethane (CFC-113) was introduced into the reactor at 10 g/h flow rate. The catalyst bed temperature increased to 216° C. upon introduction of 1,1,2-trichloro-1,2,2-trifluoroethane (CFC-113). As shown in Table 1, the conversion was 73.33% and selectivity toward ‘R-143+ recyclables’ was 83.00%. Selectivity toward undesired by-products was 17.00% in total. More specifically, selectivity was 6.27% for R-142a, 4.23% for ethane, 2.32% for R-160, 1.00% for R-143a, and 0.42% for R-152a.

In each of the tables below, the product/intermediates may comprise further recyclable components such as 1-chloro-1,1,2-trifluoroethane (HCFC-133b), and 1,2-dichloro-1,1,2-trifluoroethane (HCFC-123a), and trifluoroethylene (HFO-1123). These components are recyclable and can be eventually converted to 1,1,2-trifluoroethane (HFC-143). Referring to the process flow shown in FIG. 1, these recyclable components may be fed from collection tank 116 through line 128 back into reactor 112 for further reacting.

In each of the tables below, the by-products may comprise 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), 1-chloro-1,2-difluoroethane (HCFC-142a), and chloroethane (HCC-160). These by-products are the result of dehydrofluorination side reactions and are difficult to recycle or convert into 1,1,2-trifluoroethane (HFC-143).

TABLE 1
Product distribution for the 0.5% Pd/theta Al₂O₃ catalyst before and after dilution

| | Temp | % | Product/intermediates selectivity (%) | | | |
|---|---|---|---|---|---|---|
| Catalyst | (° C.) | Conv. | R-143 | R-133b | R-123a | Total |
| w/dilution | 216 | 73.34 | 37.33 | 30.83 | 12.72 | 83.00 |
| w/o | 225 | 98.76 | 17.48 | 28.19 | 11.12 | 58.34 |
| dilution | | | | | | |

| | Temp | % | By-product selectivity (%) | | | | | |
|---|---|---|---|---|---|---|---|---|
| Catalyst | (° C.) | Conv. | R-152a | R-170 | R-143a | R-142a | R-160 | Total |
| w/dilution | 216 | 73.34 | 0.42 | 4.23 | 1.00 | 6.27 | 2.32 | 17.00 |
| w/o | 225 | 98.76 | 0.03 | 12.74 | 1.61 | 16.65 | 5.73 | 41.66 |
| dilution | | | | | | | | |

In Table 1, the H₂ flow rate was 10 ml/min, 113 flow rate was 10 g/h, and pressure was 45 psig. For dilution, 10 ml of the 0.5% Pd/theta Al₂O₃ catalyst was diluted with 40 ml of ⅛″ SS mesh packing material.

FIG. 1 is a schematic diagram of the apparatus used in Example 1 for the conversion of 1,1,2-trichloro-1,2,2-trifluoroethane (CFC-113) to 1,1,2-trifluoroethane (HFC-143)

### Comparative Example 1

Study of Reaction with Undiluted Catalyst

This Example demonstrates conversion of 1,1,2-trichloro-1,2,2-trifluoroethane (CFC-113) to 1,1,2-trifluoroethane (HFC-143) using undiluted Pd/Al₂O₃. Reactions and product analysis were performed using the same apparatus and procedure as described in Example 1. 50 ml of the 0.5% Pd/theta Al₂O₃ catalyst was loaded into the tubular reactor and pretreated with H₂ at 200° C. for one hour. Then, while 150 ml/min of H₂ was passing through the catalyst bed at 200° C., 1,1,2-trichloro-1,2,2-trifluoroethane (CFC-113) was introduced into the reactor at 10 g/h flow rate. The catalyst bed temperature increased to 225° C. upon introduction of 1,1,2-trichloro-1,2,2-trifluoroethane (CFC-113). As shown in Table 1, the conversion was 98.76% and selectivity toward ‘R-143+ recyclables’ was 58.34%. Selectivity toward undesired by-products was 41.66% in total. More specifically, selectivity was 16.65% for R-142a, 12.74% for ethane, 5.73% for R-160, 1.61% for R-143a, and 0.03% for R-152a. This shows that Pd/theta Al₂O₃ catalyst is highly active for undesired hydro-defluorination side reactions when used undiluted.

### Example 2

Impact of Amount of Catalyst Dilution on Selectivity Towards Desired Products and/or Recyclables

This Example demonstrates that maximum selectivity toward 143+ recyclables in the process of 1,1,2-trichloro-1,2,2-trifluoroethane (CFC-113) conversion to 1,1,2-trifluoroethane (HFC-143) using Pd/alpha Al₂O₃ catalysts is achieved when the catalyst concentration is less than 50%. Catalyst concentrations represent the volume % of the catalyst with respect to the total volume of catalyst bed. In general, the total volume of the catalyst bed was 50 ml. In this example, we considered four different catalyst concentrations by mixing 10, 17, 25, and 35 ml of the 0.2% Pd/alpha Al₂O₃ catalyst with enough of ⅛″ SS mesh packing material to make a total volume of 50 ml. Reactions and product analysis were performed using the same apparatus and procedure as Example 1.

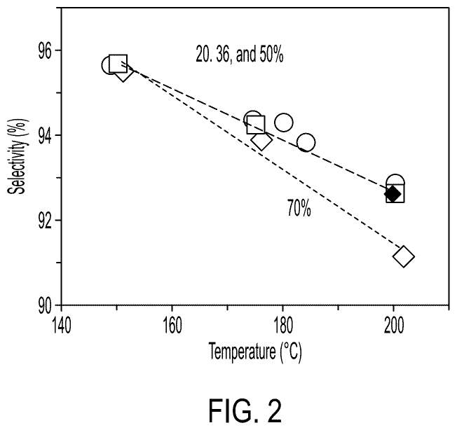

As shown in FIG. 2, total selectivity to 143+ recyclables is less for the 70% catalyst concentration compared to the catalyst concentrations below 50%; this is more evident looking at the higher temperature data. The percentages in the figure show the volume percent of the catalyst after dilution with an inert material. H₂ flow rate was 10 ml/min, 113 flow rate was 10 g/h, and pressure was 45 psig. The shown data points are average of 2-6 data points collected every two hours.

### Example 3

Conversion and Selectivity to Undesired Products with Pd/Al₂O₃ Catalysts

Example 3 demonstrates Pd/Al₂O₃ catalysts show low selectivity to the undesired by-product of R-152a in the conversion of 1,1,2-trichloro-1,2,2-trifluoroethane (CFC-113) to 1,1,2-trifluoroethane (HFC-143). Reactions and product analysis were performed using the same apparatus and procedure as described in Example 1. Six different Pd/Al₂O₃ catalysts from different sources were tested. The BET surface area of the catalysts was measured using TriStar II Micromeritics instrument. Samples were degassed before the analysis using FlowPrep 060 instrument. The measured surface area of the 0.1% Pd/gamma Al₂O₃ catalyst was 301.0 m²/g, 0.5% Pd/gamma Al₂O₃ catalyst was 302.8 m²/g, 0.3% Pd/delta Al₂O₃ catalyst was 124.4 m²/g, 0.3% Pd/theta Al₂O₃ catalyst was 40.2 m²/g, 0.5% Pd/theta Al₂O₃ catalyst was 41.1 m²/g, and 0.2% Pd/alpha Al₂O₃ catalyst was 3.9 m²/g. Table 2 shows conversion and selectivity after two hours for the conversion of 1,1,2-trichloro-1,2,2-trifluoroethane (CFC-113) to 1,1,2-trifluoroethane (HFC-143) when 10 ml of the Pd/Al₂O₃ catalyst was diluted with 40 ml of ⅛″ SS mesh packing material. The results showed that selectivity preferably toward R-152a ranges from about 0.1% to about 4% in the entire temperature range studied, from about 150° C. to about 220° C.

TABLE 2
Pd/Al₂O₃ catalysts activity and selectivity

| | Temp | % | Product/intermediates selectivity (%) | | | |
|---|---|---|---|---|---|---|
| Catalyst | (° C.) | Conv. | R-143 | R-133b | R-123a | Total |
| 0.1% Pd/gamma | 155 | 24.52 | 29.49 | 48.04 | 10.07 | 88.05 |
| Al₂O₃ | | | | | | |
| 0.5% Pd/gamma | 165 | 56.91 | 30.35 | 47.43 | 9.00 | 87.00 |
| Al₂O₃ | | | | | | |
| 0.3% Pd/delta Al₂O₃ | 205 | 71.25 | 24.67 | 38.67 | 15.71 | 80.45 |
| 0.3% Pd/theta Al₂O₃ | 152 | 38.81 | 35.89 | 47.67 | 11.13 | 94.69 |
| | 170 | 55.86 | 39.80 | 42.64 | 11.01 | 93.52 |
| | 210 | 92.08 | 42.25 | 31.26 | 11.13 | 84.89 |
| 0.5% Pd/theta Al₂O₃ | 156 | 24.10 | 31.74 | 47.59 | 14.23 | 94.15 |
| | 170 | 32.25 | 31.69 | 46.76 | 12.72 | 92.96 |
| | 216 | 73.34 | 37.33 | 30.83 | 13.83 | 83.00 |
| 0.2% Pd/alpha Al₂O₃ | 150 | 38.10 | 31.97 | 51.41 | 12.18 | 95.69 |
| | 160 | 46.61 | 31.13 | 51.58 | 11.65 | 94.64 |
| | 175 | 57.54 | 32.50 | 51.41 | 12.18 | 95.69 |
| | 200 | 82.33 | 36.61 | 41.14 | 14.59 | 92.82 |

| | Temp | % | By-product selectivity (%) | | | | | |
|---|---|---|---|---|---|---|---|---|
| Catalyst | (° C.) | Conv. | R-152a | R-170 | R-143a | R-142a | R-160 | Total |
| 0.1% Pd/gamma | 155 | 24.52 | 0.82 | 1.75 | 0.24 | 5.51 | 1.96 | 11.95 |
| Al₂O₃ | | | | | | | | |
| 0.5% Pd/gamma | 165 | 56.91 | 0.63 | 3.09 | 0.23 | 5.62 | 1.35 | 13.00 |
| Al₂O₃ | | | | | | | | |
| 0.3% Pd/delta Al₂O₃ | 205 | 71.25 | 0.10 | 5.33 | 1.29 | 7.35 | 0.31 | 19.55 |
| 0.3% Pd/theta Al₂O₃ | 152 | 38.81 | 1.70 | 1.80 | 0.36 | 0.08 | 0.08 | 5.31 |
| | 170 | 55.86 | 1.47 | 2.49 | 0.55 | 0.23 | 0.05 | 6.48 |
| | 210 | 92.08 | 0.24 | 4.94 | 1.08 | 3.36 | 0.15 | 15.11 |
| 0.5% Pd/theta Al₂O₃ | 156 | 24.10 | 2.90 | 0.83 | 0.33 | 0.25 | 0.17 | 5.85 |
| | 170 | 32.25 | 2.39 | 2.42 | 0.99 | 0.06 | 0.00 | 7.04 |
| | 216 | 73.34 | 0.42 | 4.23 | 1.00 | 6.27 | 0.50 | 17.00 |
| 0.2% Pd/alpha Al₂O₃ | 150 | 38.10 | 3.68 | 0.16 | 0.24 | 0.00 | 0.0 | 4.31 |
| | 160 | 46.61 | 3.95 | 0.36 | 0.36 | 0.00 | 0.00 | 5.36 |
| | 175 | 57.54 | 3.68 | 0.16 | 0.24 | 0.00 | 0.00 | 4.31 |
| | 200 | 82.33 | 1.66 | 2.36 | 0.85 | 0.21 | 0.05 | 7.18 |

In Table 2, 10 ml of the catalyst was diluted with 40 ml of packing material. H₂ flow rate was 10 ml/min, 113 flow rate was 10 g/h, and pressure was 45 psig.

### Comparative Example 3

Comparison of Pd/C Catalysts and Pd/Al₂O₃ Catalysts on Selectivity Towards Desired Products

Comparative Example 3 demonstrates Pd/C catalysts in general show a higher selectivity to the undesired by-product of R-152a in the process of 1,1,2-trichloro-1,2,2-trifluoroethane (CFC-113) conversion to 1,1,2-trifluoroethane (HFC-143), compared to Pd/Al₂O₃ catalysts (Example 3). Reactions and product analysis were performed using the same apparatus and procedure as described in Example 1. We explored three different Pd/C catalysts with different Pd weight loadings. Table 3 shows conversion and selectivity for the conversion of 1,1,2-trichloro-1,2,2-trifluoroethane (CFC-113) to 1,1,2-trifluoroethane (HFC-143) when 10 ml of the Pd/C catalyst was diluted with 40 ml of ⅛″ SS mesh packing material. Selectivity toward R-152a ranges from 5.51% to 11.55% in the entire temperature range studied, 150 to 250° C., which is higher than the observed selectivity over Pd/Al₂O₃ catalysts (Example 3). In addition, the maximum observed selectivity toward ‘143+ recyclables’ for the Pd/C catalysts was 93.37% for the 1% Pd/C catalyst at about 162° C.; Almost all the Pd/Al₂O₃ catalysts showed a higher overall selectivity at similar temperatures.

TABLE 3
Reactivity and product distribution results for the Pd/C catalysts

| Catalyst | Temp | % | % Product/intermediates selectivity | | | |
|---|---|---|---|---|---|---|
| (Pd/C) | (° C.) | Conv. | R-143 | R-133b | R-123a | Total |
| 0.1% | 151 | 11.13 | 27.58 | 41.15 | 17.79 | 86.52 |
| 0.5% | 153 | 14.89 | 30.42 | 37.41 | 17.80 | 87.24 |
| | 198 | 69.27 | 42.72 | 27.52 | 16.90 | 88.75 |
| 1% | 162 | 54.27 | 34.94 | 40.30 | 17.74 | 93.37 |
| | 200 | 98.36 | 46.75 | 27.22 | 14.44 | 88.79 |
| | 250 | 100 | 57.58 | 17.56 | 8.90 | 84.82 |

| Catalyst | Temp | % | % By-product selectivity | | | | | |
|---|---|---|---|---|---|---|---|---|
| (Pd/C) | (° C.) | Conv. | R-152a | R-170 | R-143a | R-142a | R-160 | Total |
| 0.1% | 151 | 11.13 | 8.98 | 0.36 | 2.88 | 0.00 | 0.00 | 13.48 |
| 0.5% | 153 | 14.89 | 11.55 | 0.27 | 0.34 | 0.00 | 0.00 | 12.76 |
| | 198 | 69.27 | 9.51 | 0.43 | 0.22 | 0.04 | 0.07 | 11.25 |
| 1% | 162 | 54.27 | 5.51 | 0.41 | 0.22 | 0.00 | 0.00 | 6.63 |
| | 200 | 98.36 | 8.77 | 1.09 | 0.32 | 0.04 | 0.14 | 11.21 |
| | 250 | 100 | 10.36 | 2.28 | 0.43 | 0.29 | 0.33 | 15.18 |

10 ml of the catalyst was diluted with 40 ml of packing material. H₂ flow rate was 10 ml/min, 113 flow rate was 10 g/h, and pressure was 45 psig.

### Example 4

### Catalyst Thermal Stability Study

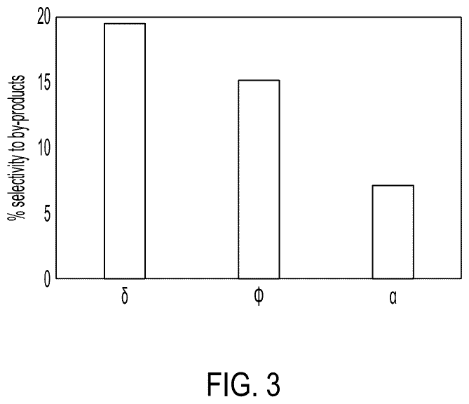

Example 4 demonstrates that alpha phase of alumina is the preferred phase for the conversion of 1,1,2-trichloro-1,2,2-trifluoroethane (CFC-113) to 1,1,2-trifluoroethane (HFC-143) using Pd/Al₂O₃ as the catalyst. Reactions and product analysis were performed using the same apparatus and procedure as described in Example 1. Six different Pd/Al₂O₃ catalysts from different sources were explored and the initial conversion/selectivity results after two hours of operation are shown in Table 2. As shown in Table 2 and FIG. 3, by-products formation follows this trend: alpha<theta<delta<gamma. Since the support stability follows the opposite trend, it appears that less stable supports are more reactive toward hydro-defluorination reactions.

FIG. 3 shows the selectivity to undesired by-products as a function of phase of the alumina support in 200-210° C. range. The alpha, theta, and delta alumina catalysts used are 0.2% Pd/alpha Al₂O₃, 0.3% Pd/theta Al₂O₃, and 0.3% Pd/delta Al₂O₃ catalysts, respectively. For these experiments, 10 ml of the catalyst was diluted with 40 ml of the packing material. H₂ flow rate was 150 ml/min, 113 flow rate was 10 g/h, and pressure was 45 psig. By-products include R-152a, R-170, R-143a, R-1132a, R-142b, R-142a, R-160, and others.

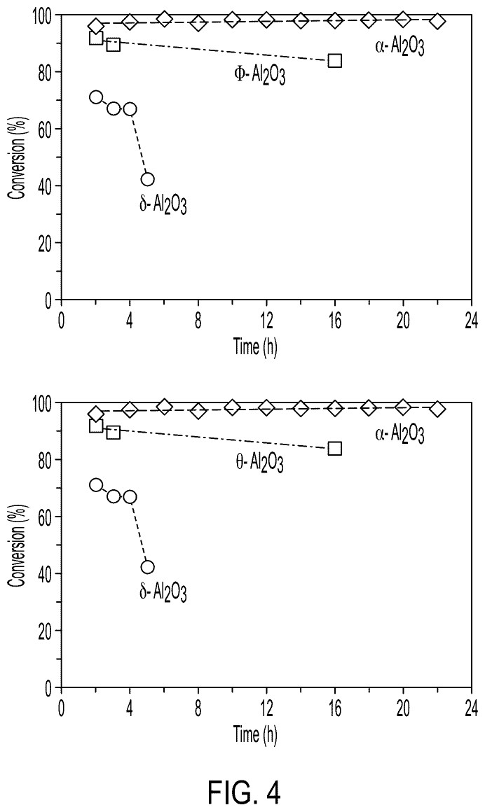

Long time stability of the catalyst is also impacted by the support phase. The catalyst using gamma Al₂O₃ as the support deactivated very quickly. The percent conversion of the 0.5% Pd/gamma Al₂O₃ catalyst changed from 56.91% after two hours to 41.07% after 8 hours at 165° C. The percent conversion of the 0.1% Pd/gamma Al₂O₃ catalyst changed from 24.52% after two hours to 19.97% after eight hours at 155° C. At a similar temperature, the 0.2% Pd/alpha Al₂O₃ catalyst did not show any sign of catalyst deactivation up to 250 hours, see Table 4. As shown in FIG. 4, the 0.3% Pd/delta Al₂O₃ deactivated very rapidly after 6 hours at 206° C. and the 0.3% Pd/theta Al₂O₃ catalyst showed 8% drop in conversion (from 92% to 84%) in 15 hours at 210° C. On the other hand, using the 0.2% Pd/alpha Al₂O₃ catalyst at 200° C., conversion increased from 95.9 to 97.9% during 22 hours of operation.

The catalysts used in FIG. 4 are 0.2% Pd/alpha Al₂O₃, 0.3% Pd/theta Al₂O₃, and 0.3% Pd/delta Al₂O₃ catalysts, respectively. The temperature was 200° C. for the 0.2% Pd/alpha Al₂O₃ catalyst, 210° C. for the 0.3% Pd/theta Al₂O₃ catalyst, and 205° C. for the Pd/delta Al₂O₃ catalyst. 10 ml of the catalyst was diluted with 40 ml of packing material for the 0.1% Pd/gamma Al₂O₃ catalyst, 0.5% Pd/gamma Al₂O₃ catalyst, 0.3% Pd/theta Al₂O₃ catalyst and 0.3% Pd/delta Al₂O₃ catalyst. 25 ml of the catalyst was diluted with 25 ml of packing material for the Pd/alpha Al₂O₃ catalyst at 200° C. H₂ flow rate was 150 ml/min, 113 flow rate was 10 g/h, and pressure was 45 psig.

Stability assessment studies were done of the 0.2% Pd/alpha Al₂O₃ catalyst at 160° C. up to 250 hours. The results are summarized in Table 4 below. The initial substrate conversion after two hours at 160° C. was 46.61% and selectivity toward ‘143+ recyclables’ was 94.64%, as shown in Table 2. Table 4 shows the performance of this catalyst under the same conditions up to 250 hours. GC samples were collected every four hours and results were averaged over 50 hours. Conversion and overall selectivity toward ‘143+ recyclables’ slowly increased over time. Selectivity toward undesired by-product of R-152a decreased over time while selectivity toward R-170, R-160, and R-142s increased with time; the net effect was a decrease in the total selectivity toward undesired by-products.

TABLE 4
Long term performance of the 0.2% Pd/alpha Al₂O₃ catalysts at
160° C. for the process of 1,1,2-trichloro-1,2,2-trifluoroethane
(CFC-113) to 1,1,2-trifluoroethane (HFC-143) conversion

| Time | % | % Product/intermediates selectivity | | | |
|---|---|---|---|---|---|
| (h) | Conv. | R-143 | R-133b | R-123a | total* |
| 0-50 | 51.51 | 31.68 | 50.35 | 12.86 | 95.03 |
| 51-100 | 53.63 | 30.93 | 50.78 | 13.55 | 95.35 |
| 101-150 | 54.44 | 30.33 | 51.27 | 13.76 | 95.45 |
| 151-200 | 55.64 | 30.01 | 51.49 | 13.93 | 95.53 |
| 201-250 | 56.44 | 31.12 | 50.74 | 13.67 | 95.63 |

| Time | % | % By-product selectivity | | | | | |
|---|---|---|---|---|---|---|---|
| (h) | Conv. | R-152a | R-170 | R-143a | R-142a | R-160 | total** |
| 0-50 | 51.51 | 3.14 | 0.78 | 0.27 | 0.04 | 0.45 | 4.97 |
| 51-100 | 53.63 | 2.05 | 1.21 | 0.27 | 0.08 | 0.73 | 4.65 |
| 101-150 | 54.44 | 1.66 | 1.36 | 0.28 | 0.10 | 0.82 | 4.55 |
| 151-200 | 55.64 | 1.42 | 1.45 | 0.29 | 0.11 | 0.89 | 4.47 |
| 201-250 | 56.44 | 1.26 | 1.40 | 0.29 | 0.14 | 0.92 | 4.37 |

In Table 4, 10 ml of the catalyst was diluted with 40 ml of packing material. H₂ flow rate was 150 ml/min, 113 flow rate was 10 g/h, and pressure was 45 psig.

### Example 5

### Conversion Study of Palladium on Alpha Alumina Catalysts

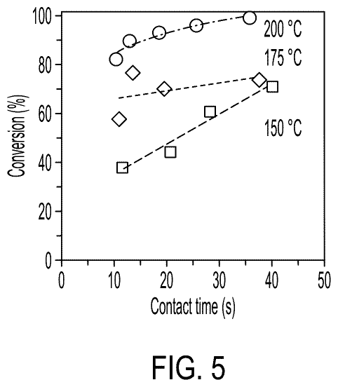

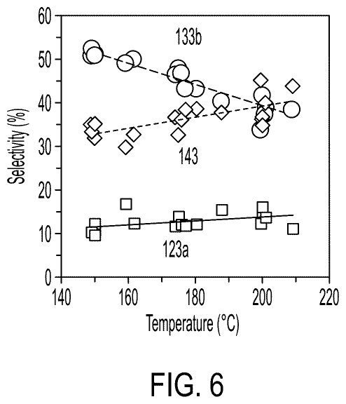

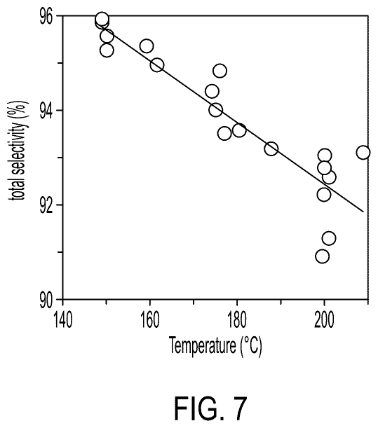

This Example demonstrates optimization of reaction conditions toward enhancing product selectivity for the conversion of 1,1,2-trichloro-1,2,2-trifluoroethane (CFC-113) to 1,1,2-trifluoroethane (HFC-143) using 0.2% Pd/alpha Al₂O₃ catalyst. Reactions and product analysis were performed using the same apparatus and procedure as described in Example 1. Table 5 shows the experiments performed. At a given temperature, substrate conversion is increasing with increase in contact time, FIG. 5. Contact time has a smaller impact on product selectivity compared to temperature. As shown in FIG. 6, considering all the experiments presented in Table 5, selectivity toward 143 and 123a increases with temperature and selectivity toward 133b decreases with temperature. In addition, selectivity toward undesired by-product increases with temperature. The net effect is a drop in overall selectivity toward ‘143+ recyclables, as shown in FIG. 7. An overall selectivity above 95% toward ‘143+ recyclables’ could be achieved at temperatures below 160° C.

TABLE 5
Process conditions, substrate conversion and product selectivity for the
process of 1,1,2-trichloro-1,2,2-trifluoroethane (CFC-113) to 1,1,2-trifluoroethane
(HFC-143) conversion using 0.2% Pd/alpha Al₂O₃ catalysts

| | | R-113 | | | | | | | | |
|---|---|---|---|---|---|---|---|---|---|---|
| Catalyst | H₂ flow | flow | | | | | Selectivity to | | | |
| volume | rate | rate | C.T. | H₂/ | Temp. | Conv. | product/intermediates | | | |
| (ml) | (ml/min) | (g/h) | (s) | 113 | (° C.) | % | R-143 | R-133b | R-123a | total |
| 10 | 150 | 10 | 11.1 | 6.8 | 161 | 60.69 | 32.66 | 50.04 | 12.24 | 94.94 |
| 10 | 300 | 20 | 5.3 | 6.8 | 180 | 73.62 | 38.6 | 43.00 | 11.98 | 93.57 |
| 10 | 300 | 25 | 5.2 | 5.5 | 188 | 67.76 | 37.75 | 40.22 | 15.21 | 93.18 |
| 10 | 300 | 30 | 5.1 | 4.6 | 200 | 76.23 | 45.07 | 33.80 | 12.03 | 90.90 |
| 10 | 300 | 25 | 5.6 | 5.5 | 159 | 30.19 | 29.6 | 48.95 | 16.80 | 95.34 |
| 10 | 150 | 10 | 11.4 | 6.8 | 150 | 38.01 | 31.97 | 51.41 | 12.18 | 95.55 |
| 10 | 150 | 10 | 10.8 | 6.8 | 175 | 57.54 | 32.5 | 47.79 | 13.71 | 94.00 |
| 10 | 150 | 10 | 10.2 | 6.8 | 200 | 82.54 | 36.36 | 41.45 | 14.41 | 92.21 |
| 18 | 150 | 10 | 20.6 | 6.8 | 149 | 44.43 | 34.91 | 50.75 | 10.20 | 95.86 |
| 18 | 150 | 10 | 19.5 | 6.8 | 174 | 70.19 | 36.66 | 46.20 | 11.53 | 94.39 |
| 18 | 150 | 10 | 18.4 | 6.8 | 201 | 93.31 | 39.67 | 39.29 | 13.62 | 92.58 |
| 25 | 150 | 10 | 28.2 | 6.8 | 149 | 61.12 | 33.36 | 52.45 | 10.09 | 95.91 |
| 25 | 300 | 20 | 13.5 | 6.8 | 176 | 76.54 | 35.99 | 46.85 | 11.98 | 94.83 |
| 25 | 300 | 30 | 12.5 | 4.6 | 209 | 82.89 | 43.71 | 38.42 | 10.98 | 93.11 |
| 25 | 150 | 10 | 25.5 | 6.8 | 200 | 95.93 | 36.71 | 41.36 | 14.96 | 93.04 |
| 25 | 300 | 20 | 12.8 | 6.8 | 200 | 90.20 | 35.03 | 41.75 | 16.01 | 92.79 |
| 35 | 150 | 10 | 40 | 6.8 | 150 | 71.09 | 35.07 | 50.79 | 9.41 | 95.27 |
| 35 | 150 | 10 | 37.6 | 6.8 | 177 | 73.82 | 38.5 | 43.16 | 11.84 | 93.50 |
| 35 | 150 | 10 | 35.7 | 6.8 | 201 | 99.36 | 40.15 | 37.37 | 13.78 | 91.29 |

In Table 5, the specified volume of the catalyst was diluted with enough of packing material to make a total volume of 50 ml. All the reactions were performed at 45 psig.

FIG. 5 shows the percent conversion of the 1,1,2-trichloro-1,2,2-trifluoroethane (CFC-113) substrate for the 0.2% Pd/alpha Al₂O₃ catalysts as a function of contact time at three different temperatures. Data correspond to the experiments presented in Table 5.

FIG. 6 shows the product selectivity for the conversion of 1,1,2-trichloro-1,2,2-trifluoroethane (CFC-113) to 1,1,2-trifluoroethane (HFC-143) using 0.2% Pd/alpha Al₂O₃ catalysts as a function of temperature. Data correspond to the experiments presented in Table 5.

FIG. 7 shows the product selectivity toward 143, 123a, and 133b in the process of 1,1,2-trichloro-1,2,2-trifluoroethane (CFC-113) conversion to 1,1,2-trifluoroethane (HFC-143) using 0.2% Pd/alpha Al₂O₃ catalysts as a function of temperature. Data correspond to the experiments presented in Table 5.

### Example 6

### Feedstock Dilution Using Inert Gas

Example 6 demonstrates beneficial effect of feed dilution using N₂ as a diluent in improving product selectivity when using Pd/Al₂O₃ as the catalyst for the conversion of 1,1,2-trichloro-1,2,2-trifluoroethane (CFC-113) to 1,1,2-trifluoroethane (HFC-143). Reactions and product analysis were performed using the same apparatus and procedure as described in Example 1. 50 ml of the 0.3% Pd/theta Al₂O₃ catalyst was loaded into the tubular reactor and pretreated with 150 ml/min of H₂ at 150° C. for one hour. Then, while the desired flow rates of 1,1,2-trichloro-1,2,2-trifluoroethane (CFC-113) and N₂ (as specified in Table 6) were passing through the catalyst bed, depending on the flow rates, the catalyst bed temperature increased to 167-174° C. upon introduction of 1,1,2-trichloro-1,2,2-trifluoroethane (CFC-113). Conversion and selectivity values are presented in Table 7. In Table 6 below, “X” represents mole fraction, assuming ideal gas behavior.

TABLE 6
Feedstock dilution effect evaluation experiments

| | R-113 | H₂ | N₂ | | | | | |
|---|---|---|---|---|---|---|---|---|
| Run | flow rate | flow rate | Flow rate | | | | N₂/ | Temp |
| # | (g/h) | ml/min | ml/min | X_{H2} | X_{R-113} | X_{N2} | R-113 | (° C.) |
| 1 | 15 | 150 | 0 | 0.82 | 0.18 | 0 | 0 | 174 |
| 2 | 10 | 200 | 22 | 0.82 | 0.09 | 0.09 | 1.0 | 169 |
| 3 | 10 | 150 | 22 | 0.77 | 0.11 | 0.11 | 1.0 | 166 |
| 4 | 10 | 150 | 55 | 0.66 | 0.10 | 0.24 | 2.5 | 167 |
| 5 | 10 | 150 | 80 | 0.59 | 0.10\| | 0.31 | 3.6 | 167 |

TABLE 7
Product distribution for the 0.3% Pd/theta
Al₂O₃ catalyst before and after feed dilution

| Run | N₂/ | % | Product/intermediates selectivity (%) | | | |
|---|---|---|---|---|---|---|
| # | R-113 | Conv. | R-143 | R-133b | R-123a | Total |
| 1 | 0 | 77.47 | 32.88 | 37.01 | 12.22 | 82.14 |
| 2 | 1.0 | 84.01 | 34.92 | 43.24 | 10.71 | 88.88 |
| 3 | 1.0 | 85.57 | 37.23 | 42.80 | 9.99 | 90.05 |
| 4 | 2.5 | 94.81 | 42.47 | 38.77 | 9.05 | 90.31 |
| 5 | 3.6 | 98.72 | 44.05 | 35.68 | 8.79 | 88.54 |

| Run | N₂/ | % | By-product selectivity (%) | | | | | |
|---|---|---|---|---|---|---|---|---|
| # | R-113 | Conv. | R-152a | R-170 | R-143a | R-142a | R-160 | Total |
| 1 | 0 | 77.47 | 0.31 | 5.55 | 0.72 | 5.46 | 2.46 | 17.85 |
| 2 | 1.0 | 84.01 | 0.22 | 3.17 | 0.40 | 4.15 | 1.50 | 11.12 |
| 3 | 1.0 | 85.57 | 0.33 | 3.16 | 0.47 | 2.67 | 1.81 | 9.95 |
| 4 | 2.5 | 94.81 | 0.56 | 4.36 | 0.61 | 1.18 | 2.03 | 9.69 |
| 5 | 3.6 | 98.72 | 0.48 | 6.13 | 0.75 | 0.76 | 2.07 | 11.46 |

In Table 7, all the experiment were performed at 45 psig. The reactor bed temperature before the introduction of organics was 150° C., and it was increased to the specified temperature in the table due to reaction exotherm.

### Comparative Example 6

### Reaction in the Absence of Feedstock Dilution

Comparative Example 6 demonstrates conversion of 1,1,2-trichloro-1,2,2-trifluoroethane (CFC-113) to 1,1,2-trifluoroethane (HFC-143) using 0.3% Pd/theta Al₂O₃ without feed dilution, Run #1, Table 6 and Table 7. Reactions and product analysis were performed using the same apparatus and procedure as described in Example 1. 50 ml of the 0.3% Pd/theta Al₂O₃ catalyst was loaded into the tubular reactor and pretreated with H₂ at 150° C. for one hour. Then, while 150 ml/min of H₂ was passing through the catalyst bed at 150° C., 1,1,2-trichloro-1,2,2-trifluoroethane (CFC-113) was introduced into the reactor at 15 g/h flow rate. The feed gas composition was 82% H₂ and 18% 1,1,2-trichloro-1,2,2-trifluoroethane (CFC-113). The catalyst bed temperature increased to 171° C. upon introduction of 1,1,2-trichloro-1,2,2-trifluoroethane (CFC-113), and then slowly increased to 174° C. over the next two hours. As shown in Table 7 (Run #1), the conversion was 77.47% and selectivity toward ‘R-143+ recyclables’ was 82.14%. Selectivity toward undesired by-products was 17.85% in total. More specifically, selectivity was 5.46% for R-142a, 5.55% for ethane, 2.46% for R-160, 0.72% for R-143a, and 0.31% for R-152a.

### Example 7

### Feedstock Dilution Using Organic Molecules

Example 7 demonstrates the beneficial effect of feedstock dilution using 1,1,2-trifluoroethane (HFC-143) as a diluent in improving product selectivity when using Pd/Al₂O₃ as the catalyst for the conversion of 1,1,2-trichloro-1,2,2-trifluoroethane (CFC-113) to 1,1,2-trifluoroethane (HFC-143).

The experiments are run the same way as described in Example 6 except for the diluent being 1,1,2-trifluoroethane (HFC-143). Similar results including lower hot spot temperatures and higher combined selectivity to 1,1,2-trifluoroethane (HFC-143), 1-chloro-1,1,2-trifluoroethane (HCFC-133b), and 1,2-dichloro-1,1,2-trifluoroethane (HCFC-123a) in the presence of each of the following diluents including HFC-41, HFC-32, HFC-23, HFC-152a, HFC-134a, HFC-125, HFC-254eb, HFC-254fb, HFC-245fa, HFC-245cb, HFC-245eb, HFC-236ea, HFC-236fa, HFC-227ea, and HFC-143 than in the absence of any of these diluents are observed. Lower selectivity to by-products including 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), 1-chloro-1,2-difluoroethane (HCFC-142a), and/or chloroethane (HCC-160) is also observed.

### Example 8

### Combining Catalyst Dilution and Feedstock Dilution

The catalyst dilution processes of Examples 1-5 are combined with the feedstock dilution processes of Examples 6 and 7. Similar or improved results including lower hot spot temperatures, higher combined selectivity to 1,1,2-trifluoroethane (HFC-143), 1-chloro-1,1,2-trifluoroethane (HCFC-133b), and 1,2-dichloro-1,1,2-trifluoroethane (HCFC-123a), and lower selectivity to by-products including 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), 1-chloro-1,2-difluoroethane (HCFC-142a), and/or chloroethane (HCC-160) is also observed.

### Aspects

Aspect 1 is a method for producing 1,1,2-trifluoroethane (HFC-143), comprising: hydrogenating 1,1,2-trichloro-1,2,2-trifluoroethane (CFC-113) by reaction with hydrogen in the presence of a catalyst to produce 1,1,2-trifluoroethane (HFC-143), the catalyst comprising: a catalytic material comprising from 0.1 to 1.0 wt. % of a catalytic metal supported on a support, based on a total weight of the catalytic metal and the support; and a diluent material, wherein the amount of catalytic material is from 5 to 70 volume percent, based on the total volume of the catalytic material and diluent material.

Aspect 2 is the method of Aspect 1, wherein the amount of catalytic material is from 10 to 50 volume percent, based on the total volume of the catalytic material and the diluent material.

Aspect 3 is the method of Aspect 1 or Aspect 2, wherein the metal comprises palladium.

Aspect 4 is the method of any one of Aspects 1-3, wherein the support comprises alumina (Al₂O₃).

Aspect 5 is the method of any one of Aspects 1-4, wherein the diluent comprises a metal or a metal alloy.

Aspect 6 is the method of any one of Aspects 1-5, wherein the support is selected from alpha alumina, delta alumina, theta alumina, and gamma alumina.

Aspect 7 is the method of any one of Aspects 1-6, wherein the support comprises alpha alumina.

Aspect 8 is the method of any one of Aspects 1-7, wherein the hydrogenation step is carried out at a temperature from about 100° C. to about 300° C.

Aspect 9 is the method of any one of Aspects 1-8, wherein the hydrogenation step is carried out at a temperature from about 150° C. to about 250° C.

Aspect 10 is the method of any one of Aspects 1-9, wherein the hydrogenation step is carried out at a pressure of from about 10 psig to about 100 psig.

Aspect 11 is the method of any one of Aspects 1-10, wherein the hydrogenation step is carried out at a ratio of 1,1,2-trichloro-1,2,2-trifluoroethane (CFC-113) to hydrogen of from about 3:1 to about 15:1.

Aspect 12 is the method of any one of Aspects 1-11, wherein the hydrogenation step achieves a selectivity to 1,1,2-trifluoroethane (HFC-143) of greater than about 20%.

Aspect 13 is the method of any one of Aspects 1-12, wherein the hydrogenation step achieves a combined selectivity to 1,1,2-trifluoroethane (HFC-143), 1-chloro-1,1,2-trifluoroethane (HCFC-133b), and 1,2-dichloro-1,1,2-trifluoroethane (HCFC-123a) of greater than about 80%.

Aspect 14 is the method of any one of Aspects 1-13, wherein the hydrogenation step achieves a combined selectivity to 1,1-difluoroethane (HFC-152a), ethane (HC-170), 1,1,1-trifluoroethane (HFC-143a) 1-chloro-1,2-difluoroethane (HCFC-142a), and chloroethane (HCC-160) of less than about 20%.

Aspect 15 is the method of any one of Aspects 1-14, wherein the hydrogenation step is carried out at a contact time of from about 1 second to about 60 seconds.

Aspect 16 is the method of any one of Aspects 1-15, further comprising the additional step of: dehydrofluorinating 1,1,2-trifluoroethane (HFC-143) in the presence of a catalyst to produce trans-1,2-difluoroethylene (HFO-1132E) and/or cis-1,2-difluoroethylene (HFO-1132Z).

Aspect 17 is the method of Aspect 16, further comprising the additional step of: isomerizing cis-1,2-difluoroethylene (HFO-1132Z) to produce trans-1,2-difluoroethylene (HFO-1132E).

Aspect 18 is a composition produced from the method of any one of Aspect 16 or Aspect 17, comprising: trans-1,2-difluoroethylene (HFO-1132E) present in an amount of at least 95 wt. %; and 1,1,1,-tritfluoroethane (HFC-143a) present in an amount of less than 5 wt. %, based on a total weight of the composition.

Aspect 19 is the composition of Aspect 18, comprising: trans-1,2-difluoroethylene (HFO-1132E) present in an amount of at least 97 wt. %; and 1,1,1,-tritfluoroethane (HFC-143a) present in an amount of less than 3 wt. %, based on a total weight of the composition.

Aspect 20 is the composition of Aspect 19, comprising: trans-1,2-difluoroethylene (HFO-1132E) present in an amount of at least 99 wt. %; and 1,1,1,-tritfluoroethane (HFC-143a) present in an amount of less than 1 wt. %, based on a total weight of the composition.

Aspect 21 is a palladium metal catalyst useful for hydrogenating 1,1,2-trichloro-1,2,2-trifluoroethane (CFC-113) by reaction with hydrogen to produce 1,1,2-trifluoroethane (HFC-143), the catalyst comprising: a catalytic material comprising from 0.1 to 1.0 wt. % of palladium metal supported on an alumina (Al₂O₃) support, based on a total weight of the catalytic metal and the support; and a diluent material, wherein the amount of catalytic material is from 5 to 70 volume percent, based on the total volume of the catalytic material and diluent material.

Aspect 22 is the catalyst of Aspect 21, wherein the amount of catalytic material is from 10 to 50 volume percent, based on the total volume of the catalytic material and the diluent material.

Aspect 23 is the catalyst of Aspect 21 or Aspect 22, wherein the support is selected from alpha alumina, delta alumina, and theta alumina.

Aspect 24 is the catalyst of any one of Aspects 21-23, wherein the support comprises alpha alumina.

Aspect 25 is the catalyst of any one of Aspects 21-24, wherein the diluent comprises a metal or a metal alloy.

Aspect 26 is a method for producing 1,1,2-trifluoroethane (HFC-143), comprising: combining 1,1,2-trichloro-1,2,2-trifluoroethane (CFC-113) with a feedstock diluent to form a reaction mixture; and reacting the 1,1,2-trichloro-1,2,2-trifluoroethane (CFC-113) in the reaction mixture with hydrogen in the presence of a catalyst to produce a product mixture.

Aspect 27 is the method of Aspect 26, wherein the product mixture comprises 1,1,2-trifluoroethane (HFC-143), and the method further comprises the additional steps of: separating the 1,1,2-trifluoroethane (HFC-143) from the product mixture; and conveying the 1,1,2-trifluoroethane (HFC-143) to the reactant mixture.

Aspect 28 is the method of Aspect 27, wherein the reaction mixture comprises a mole ratio of 1,1,2-trifluoroethane (HFC-143) to 1,1,2-trichloro-1,2,2-trifluoroethane (CFC-113), from about 0.25:1 to about 10:1.

Aspect 29 is the method of Aspect 28, wherein the reaction mixture comprises a mole ratio of 1,1,2-trifluoroethane (HFC-143) to 1,1,2-trichloro-1,2,2-trifluoroethane (CFC-113), from about 0.5:1 to about 8:1.

Aspect 30 is the method of any of Aspects 1-25, wherein the method is used concurrently with the method of any of Aspects 26-29.

What is claimed is:

1. A method for producing 1,1,2-trifluoroethane (HFC-143), comprising:
hydrogenating 1,1,2-trichloro-1,2,2-trifluoroethane (CFC-113) by reaction with hydrogen in the presence of a catalyst to produce 1,1,2-trifluoroethane (HFC-143), the catalyst comprising:
a catalytic material comprising from 0.1 to 1.0 wt. % of a catalytic metal supported on a support, based on a total weight of the catalytic metal and the support; and
a diluent material, wherein the amount of catalytic material is from 5 to 70 volume percent, based on the total volume of the catalytic material and diluent material.

2. The method of claim 1, wherein the amount of catalytic material is from 10 to 50 volume percent, based on the total volume of the catalytic material and the diluent material.

3. The method of claim 1, wherein the metal comprises palladium.

4. The method of claim 1, wherein the diluent comprises a metal or a metal alloy.

5. The method of claim 1, wherein the support is selected from alpha alumina, delta alumina, and theta alumina.

6. The method of claim 1, wherein the hydrogenation step is carried out at a temperature from about 100° C. to about 300° C.

7. The method of claim 1, wherein the hydrogenation step is carried out at a mole ratio of hydrogen to 1,1,2-trichloro-1,2,2-trifluoroethane (CFC-113) of from 3:1 to 15:1.

8. The method of claim 1, wherein the hydrogenation step achieves a selectivity to 1,1,2-trifluoroethane (HFC-143) of greater than about 20%.

9. The method of claim 1, wherein the hydrogenation step achieves a combined selectivity to 1,1,2-trifluoroethane (HFC-143), 1-chloro-1,1,2-trifluoroethane (HCFC-133b), and 1,2-dichloro-1,1,2-trifluoroethane (HCFC-123a) of greater than about 80%.

10. The method of claim 1, wherein the hydrogenation step achieves a combined selectivity to 1,1-difluoroethane (HFC-152a), ethane (HC-170), 1,1,1-trifluoroethane (HFC-143a) 1-chloro-1,2-difluoroethane (HCFC-142a), and chloroethane (HCC-160) of less than about 20%.

11. The method of claim 1, wherein the hydrogenation step is carried out at a contact time of from about 1 second to about 60 seconds.

12. The method of claim 1, further comprising the additional step of:
dehydrofluorinating 1,1,2-trifluoroethane (HFC-143) in the presence of a catalyst to produce trans-1,2-difluoroethylene (HFO-1132E) and/or cis-1,2-difluoroethylene (HFO-1132Z).

13. A composition produced from the method of claim 1, comprising:
trans-1,2-difluoroethylene (HFO-1132E) present in an amount of at least 95 wt. %; and
1, 1,1,-tritfluoroethane (HFC-143a) present in an amount of less than 5 wt. %, based on a total weight of the composition.

14. A palladium metal catalyst useful for hydrogenating 1,1,2-trichloro-1,2,2-trifluoroethane (CFC-113) by reaction with hydrogen to produce 1,1,2-trifluoroethane (HFC-143), the catalyst comprising:
a catalytic material comprising from 0.1 to 1.0 wt. % of palladium metal supported on an alumina (Al₂O₃) support, based on a total weight of the catalytic metal and the support; and
a diluent material, wherein the amount of catalytic material is from 5 to 70 volume percent, based on the total volume of the catalytic material and diluent material.

15. The catalyst of claim 14, wherein the amount of catalytic material is from 10 to 50 volume percent, based on the total volume of the catalytic material and the diluent material.

16. The catalyst of claim 14, wherein the support is selected from alpha alumina, delta alumina, and theta alumina.

17. The catalyst of claim 14, wherein the diluent comprises a metal or a metal alloy.

18. A method for producing 1,1,2-trifluoroethane (HFC-143), comprising:
combining 1,1,2-trichloro-1,2,2-trifluoroethane (CFC-113) with a feedstock diluent to form a reaction mixture; and
reacting the 1,1,2-trichloro-1,2,2-trifluoroethane (CFC-113) in the reaction mixture with hydrogen in the presence of a catalyst to produce a product mixture.

19. The method of claim 18, wherein the product mixture comprises 1,1,2-trifluoroethane (HFC-143), and the method further comprises the additional steps of:
separating the 1,1,2-trifluoroethane (HFC-143) from the product mixture; and
conveying the 1,1,2-trifluoroethane (HFC-143) to the reactant mixture.

20. The method of claim 19, wherein the dilution composition comprises a mole ratio of 1,1,2-trifluoroethane (HFC-143) to 1,1,2-trichloro-1,2,2-trifluoroethane (CFC-113), from about 0.25:1 to about 10:1.
